(19) United States
(12) Patent Application Publication
(10) Pub. No.: US 2016/0174878 A1
(43) Pub. Date: Jun. 23, 2016

(54) System and Method for Measuring Skin Firmness

(71) Applicant: Avon Products, Inc., Suffern, NY (US)

(72) Inventors: Gary Lee Grove, Media, PA (US); Eugene Lee Wildman, IV, Media, PA (US); Jonn Paul Damia, Media, PA (US); Timothy Paul Houser, JR., Broomhall, PA (US); LisaMarie DiNatale, White Plains, NY (US); Keith David Ertel, Raleigh, NC (US)

(21) Appl. No.: 14/804,822

(22) Filed: Jul. 21, 2015

Related U.S. Application Data
(60) Provisional application No. 62/095,261, filed on Dec. 22, 2014.

(51) Int. Cl. A61B 5/11; A61B 5/00

(52) U.S. Cl. CPC A61B 5/1128; A61B 5/7271; A61B 5/442; A61B 5/742

(57) ABSTRACT

Disclosed is a method and system including a processor instructing a restraining device (e.g., a chair) to move the patient at a predetermined sweep rate in a predetermined motion. The patient has adhesive dots positioned on the patient's body. The processor obtains images (e.g., a video) during the predetermined motion. The processor analyzes the images to determine an amount of movement of the adhesive dots on the patient's body.

### CROSS-REFERENCE TO RELATED APPLICATIONS

This patent application claims priority to U.S. patent application Ser. No. 62/095,261, filed on Dec. 22, 2014. The entirety of the aforementioned application is incorporated herein in its entirety by reference.

### FIELD

The present disclosure relates to an apparatus, system, and method(s) for measuring skin firmness, and more specifically to objectively measuring skin firmness (e.g., on an individual's face) using Dynamic Object Tracking

### BACKGROUND

As an individual ages and/or their body mass index (BMI) changes, the firmness of their skin (e.g., on their face) also changes. For example, when a person ages, the skin on their face may begin to sag. The amount of sagging usually varies from individual to individual. When a person is young, they often do not have much sagging of their skin. As that person gets older, the collagen and elastic fibers in the skin degrade and the skin consequently loses tautness, manifesting in a greater degree of sagging.

Skin sagging also may result from weight loss. If a person loses substantial amounts of weight, their skin may not recover the degree of taughtness it had prior to the weight loss. It is difficult to quantify the degree of sagging skin. Companies develop products to reduce or minimize the sagging of the skin, but measuring the efficacy of these products is often difficult due to a lack of objective measurement tools of appropriate sensitivity.

One existing technique to measure skin firmness is using a device such as a dermal torque meter, suction cup, cutometer, gas bearing electrodynometer, etc. attached to the skin. These devices, however, have a noticeable mass and will deform the skin when attached. As a result, these devices will likely influence the results of the measurements, and, depending on the degree of sagging, may lack the sensitivity to provide meaningful data.

Another conventional technique to measure skin firmness is to compare “before and after” photographs. Comparing photographs of the face to photo-document changes in facial sagging due to treatment is often performed. For example, reference marks can be drawn on the face and then frontal photographs can be taken with the panelist in a supine and sitting position. Since the gravitational forces pull on the face in different directions in these positions, the degree to which the marks on the cheek differ in these two positions is directly related to facial firmness.

### SUMMARY

In one aspect, a method and system includes a processor instructing a restraining device (e.g., a chair) that can move in a controlled range of motion and that restricts movement of a patient to move the patient at a predetermined sweep rate in a predetermined motion. The patient has a plurality of dots positioned (e.g., with an adhesive) at predetermined locations on the patient's body (e.g., on the patient's face). The processor obtains a plurality of images (e.g., a video) during the predetermined motion. A processor (which may be the same processor or a separate processor)_{—} then analyzes the images to determine an amount of movement of the adhesive dots on the patient's body.

In another aspect, a method and system includes a processor instructing a restraining device (e.g., a chair) that can move in a controlled range of motion and that restricts movement of a patient to move the patient at a predetermined sweep rate in a predetermined motion. The patient has a plurality of dots positioned (e.g., with an adhesive) at predetermined locations on the patient's body (e.g., on the patient's face). A processor obtains baseline images (e.g., a video) during the predetermined motion. Following a time interval (e.g., one week, two weeks, four weeks, eight weeks, or more), during which time the subject may employ a treatment to address skin sagging, the processor instructs the chair to move the patient again at the predetermined sweep rate in the predetermined motion. A processor obtains a post-treatment video during the predetermined motion. A processor analyzes the post-treatment video and the baseline video to determine an amount of movement of the adhesive dots on the patient's body.

In one embodiment, the adhesive dots are positioned at predetermined locations on the patient's body (e.g., via an adhesive backing) The positioning of the adhesive dots at the predetermined locations on the patient's body can further include positioning the adhesive dots on both the left side and the right side of the patient's face (e.g., symmetrically). In one embodiment, adhesive dots can also be placed on the nose of the patient. Since skin sagging is not typical on the nose, it can serve as a frame of reference against which displacement of other dots can be measured. In one embodiment, after a certain predetermined time period has elapsed following a previous or baseline measurement, the corresponding adhesive dots may be reapplied to the patient's body for subsequent measurements. To reapply the dots in the same position as the original positions, a grid may be projected onto the patient's body.

To obtain the images, cameras (e.g., video cameras) are positioned on or near the restraining device (e.g., a chair) for example, through placement on a bar (which bar may or may not be attached to the chair). The bar may include a contact point (e.g., a plate) for the patient to engage (e.g., bite onto) to maintain a fixed relationship between the video cameras and the patient's face regardless of how the chair is moved.

The chair may be, for example, a modified car seat attached to a design frame that includes a bell crank linkage. In one embodiment, the chair includes a linear servo actuator attached to the bell crank linkage. The speed and direction of motion of the linear servo actuator can be controlled electronically via the processor.

In another aspect, a method includes positioning adhesive dots at predetermined locations on a patient's body of a patient, positioning the patient in a chair that can move in a controlled range of motion, the chair restricting movement of the patient, instructing, by a computing device, the chair to move at a predetermined sweep rate in a predetermined motion, obtaining, by video cameras, a video during the predetermined motion. The method further includes analyzing the video by the computing device to determine an amount of movement of the adhesive dots on the patient's body.

In another aspect, a method includes positioning adhesive dots at predetermined locations on a patient's body of a patient, positioning the patient in a chair that can move in a controlled range of motion, the chair restricting movement of the patient, instructing, by a computing device and/or processor, the chair to move at a predetermined sweep rate in a predetermined motion, obtaining, by video cameras, a baseline video during the predetermined motion. Subsequent to obtaining the baseline reading, in one embodiment, a skin product is applied to the patient's body (e.g., face). After a treatment regimen, the adhesive dots are reapplied at the corresponding predetermined locations. The method further includes instructing, by the computing device, the chair to move at the predetermined sweep rate in the predetermined motion, obtaining, by the video cameras, a post-treatment video during the predetermined motion after the applying of the skin product, and analyzing, by the computing device, the post-treatment video and the baseline video to determine an amount of movement of the adhesive dots on the patient's body.

These and other aspects and embodiments will be apparent to those of ordinary skill in the art by reference to the following detailed description and the accompanying drawings.

### BRIEF DESCRIPTION OF THE DRAWINGS

In the drawing figures, which are not to scale, and where like reference numerals indicate like elements throughout the several views:

FIG. 1A shows a flowchart illustrating steps performed to objectively measure skin firmness on the face in accordance with an embodiment of the present disclosure;

FIG. 1B shows a flowchart illustrating steps performed to objectively measure skin firmness on the face in accordance with another embodiment of the present disclosure;

FIG. 1C shows a flowchart illustrating steps performed to demonstrate the effectiveness of the measurement of skin firmness on the face in accordance with an embodiment of the present disclosure;

FIG. 1D shows a flowchart illustrating steps performed to demonstrate the effectiveness of the measurement of skin firmness on the patient's body in accordance with an embodiment of the present disclosure;

FIG. 2 shows a side view image of a patient's face with an adhesive dot being applied to the patient's face in accordance with an embodiment of the present disclosure;

FIG. 3A shows a perspective view of a chair that can move in a controlled range of motion that the patient sits on to measure the patient's skin firmness in accordance with an embodiment of the present disclosure;

FIG. 3B shows a perspective view of a patient in a chair that can move in a controlled range of motion, the patient biting on a bite plate of a bar in accordance with an embodiment of the present disclosure;

FIG. 4A is a perspective view of an embodiment of a bar that is mounted to the chair of FIG. 3B and a side view of a patient engaging the bite plate of the bar;

FIG. 4B is a perspective view of an embodiment of a bar that may be mounted to the chair of FIG. 3A or 3B; the bar includes a bite plate in accordance with an embodiment of the present disclosure;

FIG. 5 is a graphical representation of the trajectory of each dot during the tilt excursion in accordance with an embodiment of the present disclosure;

FIG. 6 is a tabular representation of a randomization schedule in accordance with an embodiment of the present disclosure;

FIG. 7 is a tabular representation of DOT (Dynamic Object Tracking) analysis results in accordance with an embodiment of the present disclosure;

FIG. 8 shows the tabular representation of FIG. 7 ordered by age from youngest to oldest in accordance with an embodiment of the present disclosure;

FIG. 9 is a graphical representation of the mean DOT value plotted against age cohort in accordance with an embodiment of the present disclosure;

FIG. 10 is a graphical representation of the mean DOT value plotted against age in years in accordance with an embodiment of the present disclosure;

FIG. 11 shows a graphical representation of mean DOT value plotted against age cohort for pre control, pre treated, post control, and post treated for a test product in accordance with an embodiment of the present disclosure; and

FIG. 12 is a block diagram illustrating an internal architecture of a computer in accordance with an embodiment of the present disclosure.

### DESCRIPTION OF EMBODIMENTS

Embodiments are now discussed in more detail referring to the drawings that accompany the present application. In the accompanying drawings, like and/or corresponding elements are referred to by like reference numbers.

Various embodiments are disclosed herein; however, it is to be understood that the disclosed embodiments are merely illustrative of the disclosure that can be embodied in various forms. In addition, each of the examples given in connection with the various embodiments is intended to be illustrative, and not restrictive. Further, the figures are not necessarily to scale, some features may be exaggerated to show details of particular components (and any size, material and similar details shown in the figures are intended to be illustrative and not restrictive). Therefore, specific structural and functional details disclosed herein are not to be interpreted as limiting, but merely as a representative basis for teaching one skilled in the art to variously employ the disclosed embodiments.

The present invention is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer, special purpose computer, ASIC, or other programmable data processing apparatus, such that the instructions, which execute via the processor of the computer or other programmable data processing apparatus, implements the functions/acts specified in the block diagrams or operational block or blocks.

In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved. Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

Throughout the specification and claims, terms may have nuanced meanings suggested or implied in context beyond an explicitly stated meaning Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

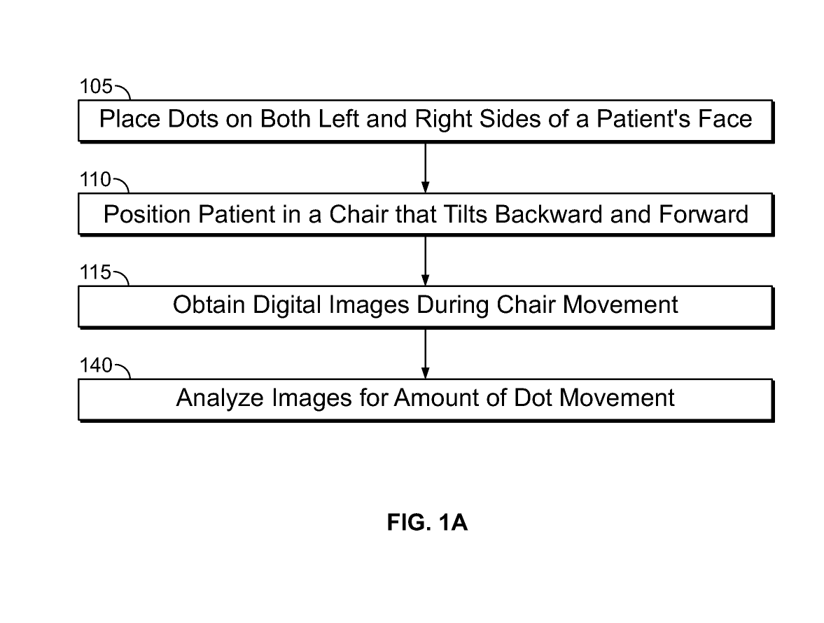

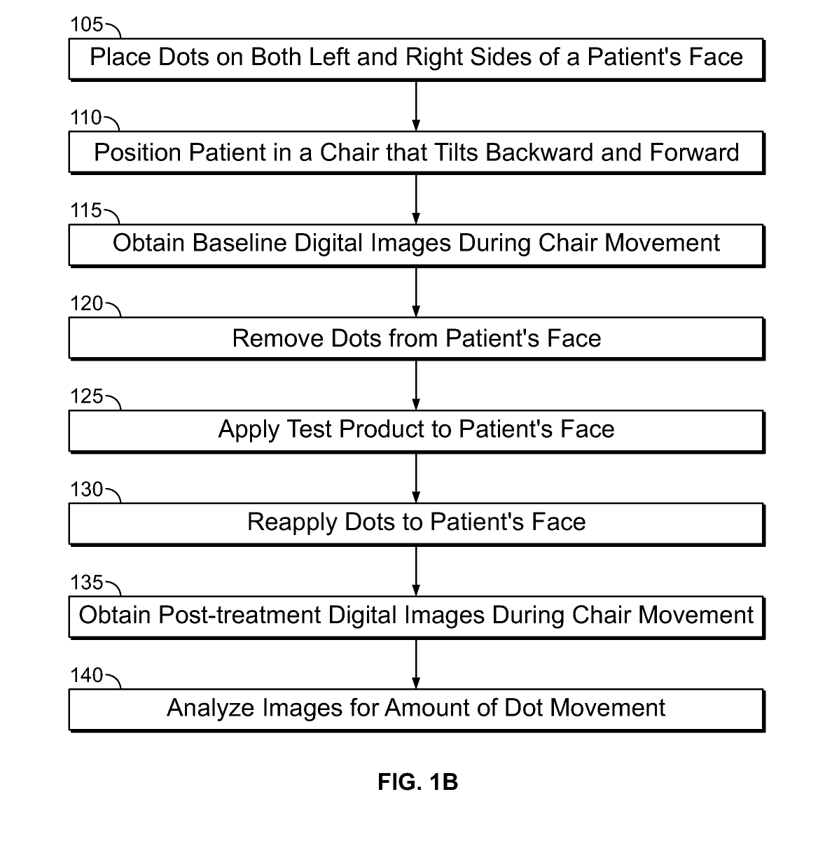

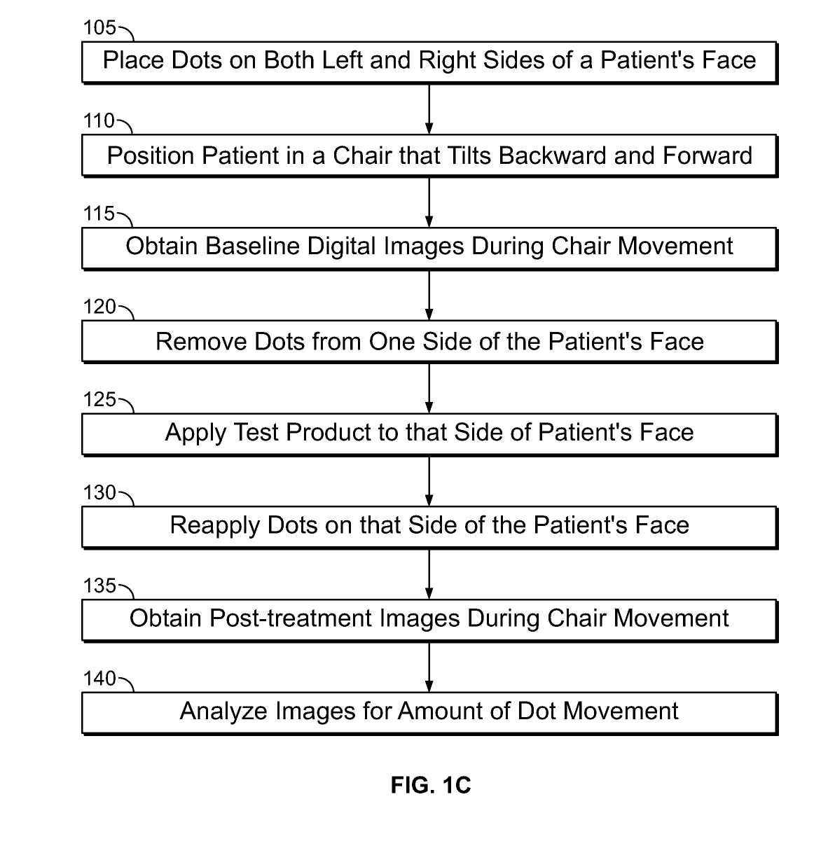

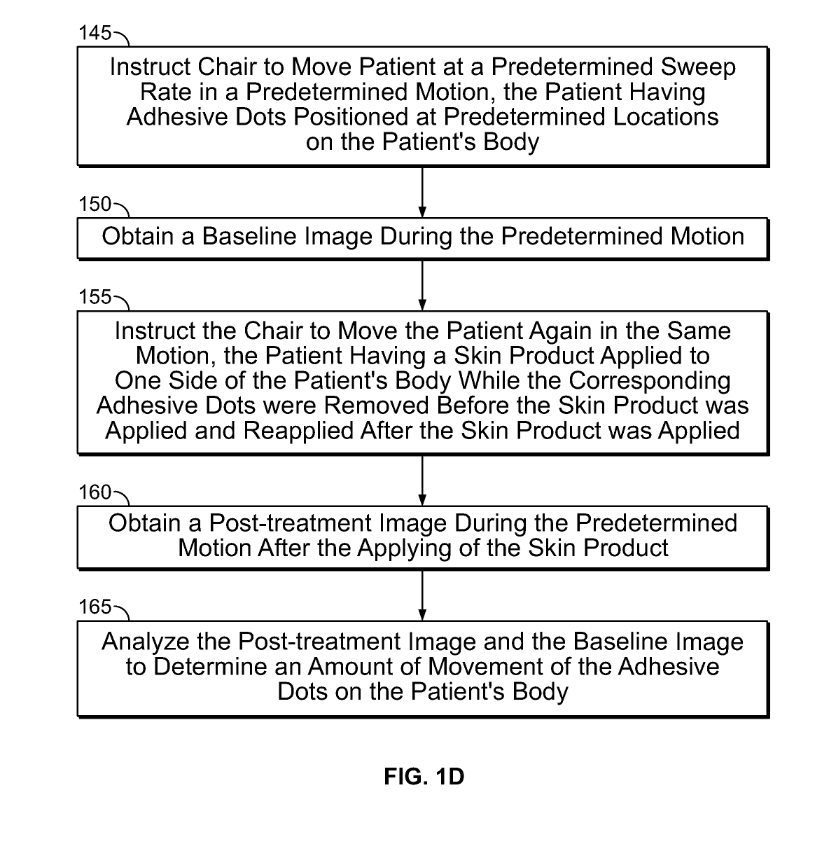

FIG. 1A shows a flowchart illustrating steps performed to objectively measure skin firmness on the face according to an embodiment of the invention. Although described as measuring the skin firmness of the face, the skin firmness of any part of a person's body can be measured using these steps. In one embodiment, this non-contact technique uses DOT (Dynamic Object Tracking) Analysis to measure the skin firmness (or skin sagging). In one embodiment, a plurality (e.g., two, three, four, or five) of adhesive dots are placed in a predetermined configuration (e.g., in a diamond pattern) on both the left and right sides of the face (Step 105). The dots may be placed symmetrically on each side of the face. In one embodiment, a reference dot is placed on each side of the nose to detect head movement (if any) relative to one or more cameras.

As described in more detail below, the patient is then positioned in a restraining device (e.g., a chair) that tilts backward and forward (Step 110). Although described as placing the dots on the patient before positioning the patient in the chair, in another embodiment the dots are placed on the patient's face after the patient sits in the chair. The chair can be swept from an upright position to fully laying back, and then returning to the initial seated position. In one embodiment, the chair can move in other directions, such as from side to side, upwards and downwards, and any combination of these directions. In one embodiment, the chair includes video cameras mounted on a bar connected to the chair on each side of the location for the patient's face. In one embodiment, the cameras are mounted at the position of the patient's cheeks. The position of the cameras may be adjustable or fixed.

In some embodiments, digital images (e.g., digital videos) are then obtained (Step 115). To obtain these videos, in some embodiments the patient is secured to the chair and videos may be taken simultaneously of the left and right sides of the face with the motorized chair moving in a predetermined path (e.g., backwards from an upright seated position to fully laying back and then moving back to the upright position). The speed of the chair movement can be either predetermined or adjustable by a technician or operator.

The videos are then analyzed for the amount of dot movement (Step 140). In one embodiment, the videos are analyzed for the amount of dot movement through each position. The movement of the dots may be tracked as captured in a video clip to assess not only the displacement of the marker dots at the two extremes in position but every degree along the way.

FIG. 1B shows another embodiment of a flowchart illustrating steps performed to objectively measure skin firmness on the face. A plurality of adhesive dots are placed on the face (Step 105), followed by positioning in a tilting chair (Step 110), as described above. In this embodiment, a baseline video is then obtained (Step 115). Once this baseline video has been obtained, the adhesive dots are removed (Step 120). After obtaining the baseline video, the patient may undergo a treatment to improve skin firmness. A product whose effects on skin firmness and/or sagging are being tested (“test product”) may be applied (Step 125). The treatment may be one that is immediately effective (e.g., Botox injection) or may be a treatment regiment requiring some time (e.g., several weeks) to achieve noticeable results (e.g., retinol). Upon completion of the desired treatment with a test product, adhesive dots are reapplied to the patient's face (Step 130). In one embodiment, the dots are applied to the same location post treatment using ghosting digital imaging software. The chair is again moved in the same pattern, and at the same speed. Post-treatment digital videos are taken (Step 135), and the videos are then analyzed for the amount of dot movement (Step 140) as described above.

In another embodiment (not shown in the figures), the following steps may be performed in the absence of a restraining means and by utilizing the subject's attempt to make a particular movement so as to objectively measure skin firmness on the face. A plurality of adhesive dots are placed on the face. A baseline video may then be obtained, during which video the patient may be asked to make a particular movement (e.g., smiling). Other movement by the patient may be minimized by the use of a restraining device during this process. Once this baseline video has been obtained, the adhesive dots are removed. After obtaining the baseline video, the patient may undergo a treatment to improve skin firmness. A product whose effects on skin firmness and/or sagging are being tested (“test product”) may be applied. The treatment may be one that is immediately effective (e.g., Botox injection) or may be a treatment regiment requiring some time (e.g., several weeks) to achieve noticeable results (e.g., retinol). Upon completion of the desired treatment with a test product, adhesive dots are reapplied to the patient's face. In one embodiment, the dots are applied to the same location post treatment using ghosting digital imaging software. Post-treatment digital videos are taken, again asking the patient to repeat the same movement made during the baseline test and with the use of optional restraint to prevent additional movement, and the videos are then analyzed for the amount of dot movement as described above.

The effectiveness of the invention for quantifying skin firmness was validated by the following experiments, the steps of which are shown in FIG. 1C and 1D.

A basic experimental design is shown in FIG. 1C. This protocol may be useful for longitudinal studies to evaluate effectiveness of anti-aging treatments. In one embodiment, this non-contact technique uses DOT (Dynamic Object Tracking) Analysis to measure the skin firmness (or skin sagging). In one embodiment, a plurality (e.g., two, three, four, or five) of adhesive dots are placed in a predetermined configuration (e.g., in a diamond pattern) on both the left and right sides of the face (Step 105). The dots are placed symmetrically on each side of the face. In one embodiment, a reference dot is placed on each side of the nose to detect head movement (if any) relative to one or more cameras. The patient is then positioned in a chair that tilts backward and forward (Step 110), as described above. Baseline digital videos are then obtained (Step 115) as described above.

Once this baseline video has been obtained, the cheek dots from one side of the face (randomized) were removed (Step 120). The nose dot was not removed so as to provide a frame of reference for subsequent measurements. A test product is then applied to that side of the patient's face (the side with the dots removed) (Step 125). The product may be a product that produces an immediate tightening benefit to the skin. In one embodiment, the test product is allowed to dry for a certain specified period of time (e.g., for ten minutes). The cheek dots are then reapplied on that side of the patient's face (Step 130). In one embodiment, the dots are applied to the same location post treatment using ghosting digital imaging software. The chair is again moved in the same pattern, and at the same speed. Post-treatment digital videos are taken (Step 135).

The videos are then analyzed for the amount of dot movement (Step 140). In one embodiment, the trajectory of or path traveled by the dot is analyzed by comparing the post-treatment videos with the baseline videos. In one embodiment, the videos are analyzed for the amount of dot movement through each position (e.g., for each age group) to determine if differences in facial sagging could be captured between age groups and to determine if treatment differences could be captured. The movement of the dots may be tracked as captured in a video clip to assess not only the displacement of the marker dots at the two extremes in position but every degree along the way.

Another experimental design is shown in FIG. 1D. This protocol may be useful for longitudinal studies to evaluate effectiveness of anti-aging treatments. In one embodiment, a processor of a computing device instructs a chair that can move in a controlled range of motion and that restricts movement of a patient to move the patient at a predetermined sweep rate in a predetermined motion, where the patient has adhesive dots positioned at predetermined locations on the patient's body (Step 145). The processor obtains a baseline video during the predetermined motion (Step 150). After obtaining the baseline video, the patient may undergo a treatment to improve skin firmness. The treatment may be one that is immediately effective (e.g., Botox injection) or may be a treatment regiment requiring several weeks to achieve noticeable results (e.g., retinols). After treatment, the patient is again seated in the chair and the processor then instructs the chair to move the patient again at the predetermined sweep rate in the predetermined motion (Step 155). The processor obtains a post-treatment video during the predetermined motion after the applying of the skin product (Step 160). The processor analyzes the post-treatment video and the baseline video to determine an amount of movement of the adhesive dots on the patient's body (Step 165).

In one embodiment, the experiments shown in FIGS. 1C and 1D were performed on patients (e.g., females with a clean face and no makeup initially applied to their face) between the ages of 18-25 with no apparent facial sagging, patients between the ages of 35-45 with mild-moderate facial sagging, and patients between the ages of 60-70 with moderate-severe facial sagging. Although specific age groups are referenced, any age group can be utilized. The facial sagging screening may be a tactile assessment.

A computing device or processor may be capable of sending or receiving signals, such as via a wired or wireless network, or may be capable of processing or storing signals, such as in memory as physical memory states. In one embodiment, a single computing device or processor may be utilized. In another embodiment, a plurality of computing device(s) or processor(s) may be used. For example, a series of computer device(s) or processor(s) adapted to perform discrete tasks (such as image analysis) may be utilized. In another embodiment, the computing device or processor may operate as a server. Thus, devices capable of operating as a server may include, as examples, dedicated rack-mounted servers, desktop computers, laptop computers, set top boxes, integrated devices combining various features, such as two or more features of the foregoing devices, or the like. Servers may vary widely in configuration or capabilities, but generally a server may include one or more central processing units and memory. A server may also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

Examples of devices that may operate as a server include desktop computers, multiprocessor systems, microprocessor-type or programmable consumer electronics, etc. A server may provide a variety of services that include, but are not limited to, web services, third-party services, audio services, video services, email services, instant messaging (IM) services, SMS services, MMS services, FTP services, voice over IP (VOIP) services, calendaring services, photo services, social media services, or the like. Examples of content may include text, images, audio, video, or the like, which may be processed in the form of physical signals, such as electrical signals, for example, or may be stored in memory, as physical states, for example. In one embodiment, a server hosts or is in communication with a database.

A network may couple devices so that communications may be exchanged, such as between a server and a client device or other types of devices, including between wireless devices coupled via a wireless network, for example. A network may also include mass storage, such as network attached storage (NAS), a storage area network (SAN), or other forms of computer or machine readable media, for example. A network may include the Internet, one or more local area networks (LANs), one or more wide area networks (WANs), wire-line type connections, wireless type connections, or any combination thereof. Likewise, sub-networks, such as may employ differing architectures or may be compliant or compatible with differing protocols, may interoperate within a larger network. Various types of devices may, for example, be made available to provide an interoperable capability for differing architectures or protocols. As one illustrative example, a router may provide a link between otherwise separate and independent LANs.

A communication link or channel may include, for example, analog telephone lines, such as a twisted wire pair, a coaxial cable, full or fractional digital lines including T1, T2, T3, or T4 type lines, Integrated Services Digital Networks (ISDNs), Digital Subscriber Lines (DSLs), wireless links including satellite links, or other communication links or channels, such as may be known to those skilled in the art. Furthermore, a computing device or other related electronic devices may be remotely coupled to a network, such as via a telephone line or link, for example.

A wireless network may couple client devices with a network. A wireless network may employ stand-alone ad-hoc networks, mesh networks, Wireless LAN (WLAN) networks, cellular networks, or the like. A wireless network may further include a system of terminals, gateways, routers, or the like coupled by wireless radio links, or the like, which may move freely, randomly or organize themselves arbitrarily, such that network topology may change, at times even rapidly. A wireless network may further employ a plurality of network access technologies, including Long Term Evolution (LTE), WLAN, Wireless Router (WR) mesh, or 2nd, 3rd, or 4th generation (2G, 3G, or 4G) cellular technology, or the like. Network access technologies may enable wide area coverage for devices, such as client devices with varying degrees of mobility, for example.

For example, a network may enable RF or wireless type communication via one or more network access technologies, such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, or the like. A wireless network may include virtually any type of wireless communication mechanism by which signals may be communicated between devices, such as a client device or a computing device, between or within a network, or the like.

In one embodiment and as described herein, the chair includes a processor. The processor may also be part of a mobile device such as a smartphone. In another embodiment, the processor is part of a tablet, laptop, desktop computer, or other computing device.

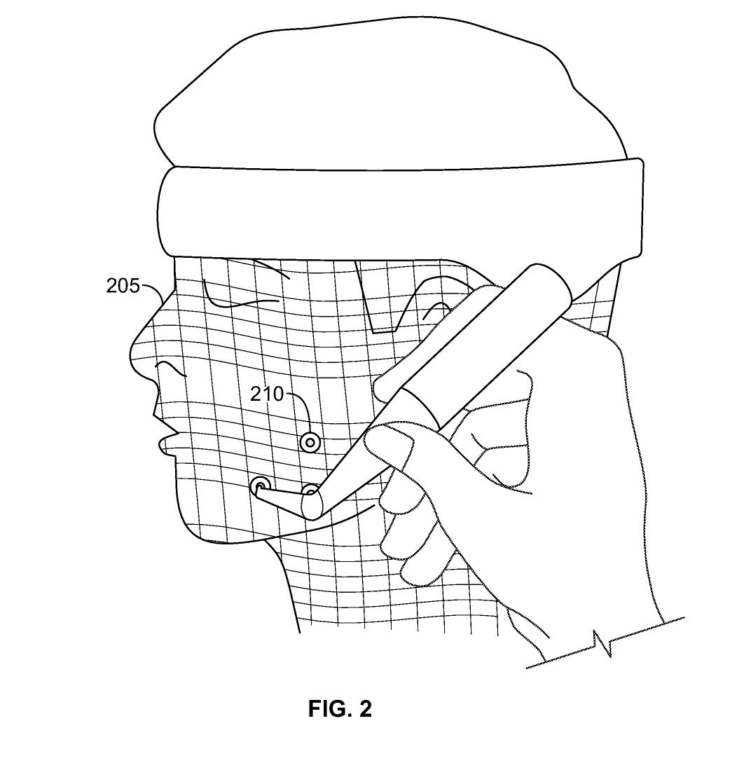

FIG. 2 shows an embodiment of a side view image of a patient's face 205 with an adhesive dot 210 being applied to the patient's face 205. As described above, five adhesive dots 210 are placed in a diamond pattern on both the left and right sides of the face, as well as a reference dot on each side of the nose to detect head movement (if any) relative to the camera. In one embodiment, dot placement and replacement of each dot to the same location post treatment is accomplished by projecting the dot pattern onto the cheek using a small projector. The projector can project a grid on the side of the face, and can be aligned using the major X and Y axis. The dots are then placed on the cheek in the spots indicated by the dots projected on the grid using ghosting digital imaging software. Dot placement and replacement of each dot to the same location post treatment can be accomplished by a pattern of five dots, focusing on the center dot and ensuring that the outer four corners are all in the same focal plane relative to that camera.

Although one could mark the skin by using a felt tip pen or surgical scribe, as is typically done with other methods in which the displacements are measured from static pre and post photos as described above, this method uses a target that can be autonomously recognized in the image frame without further operator interaction. In one embodiment, a green dot surrounded by a white background makes a good target in this application. The color(s) and/or size of the dots can vary.

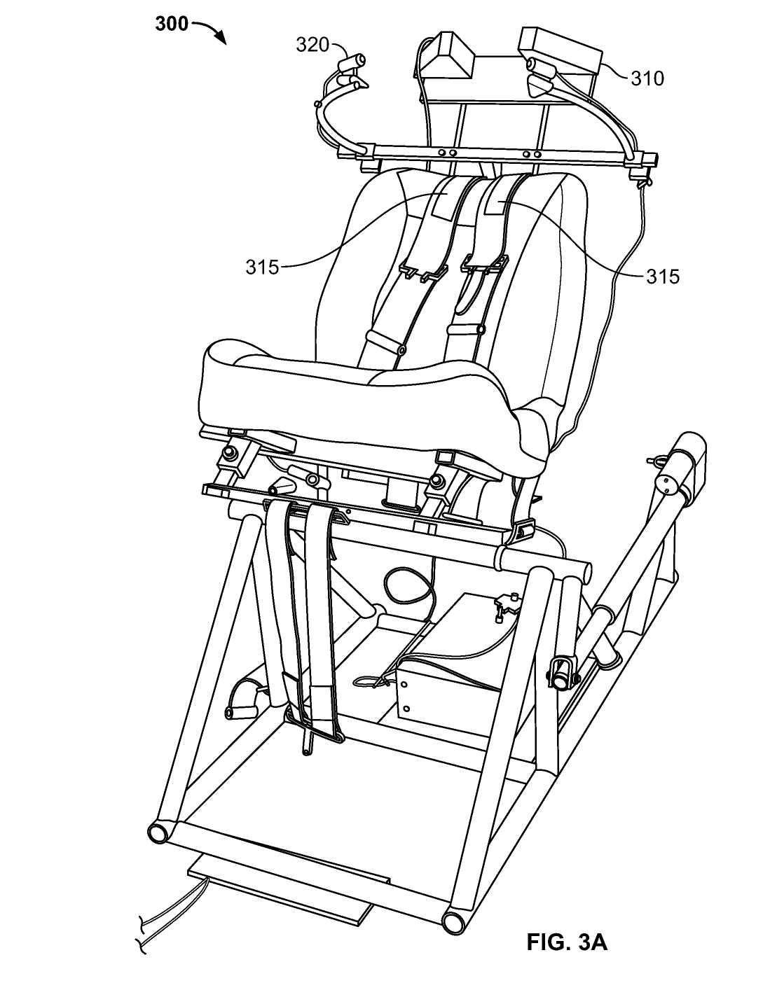

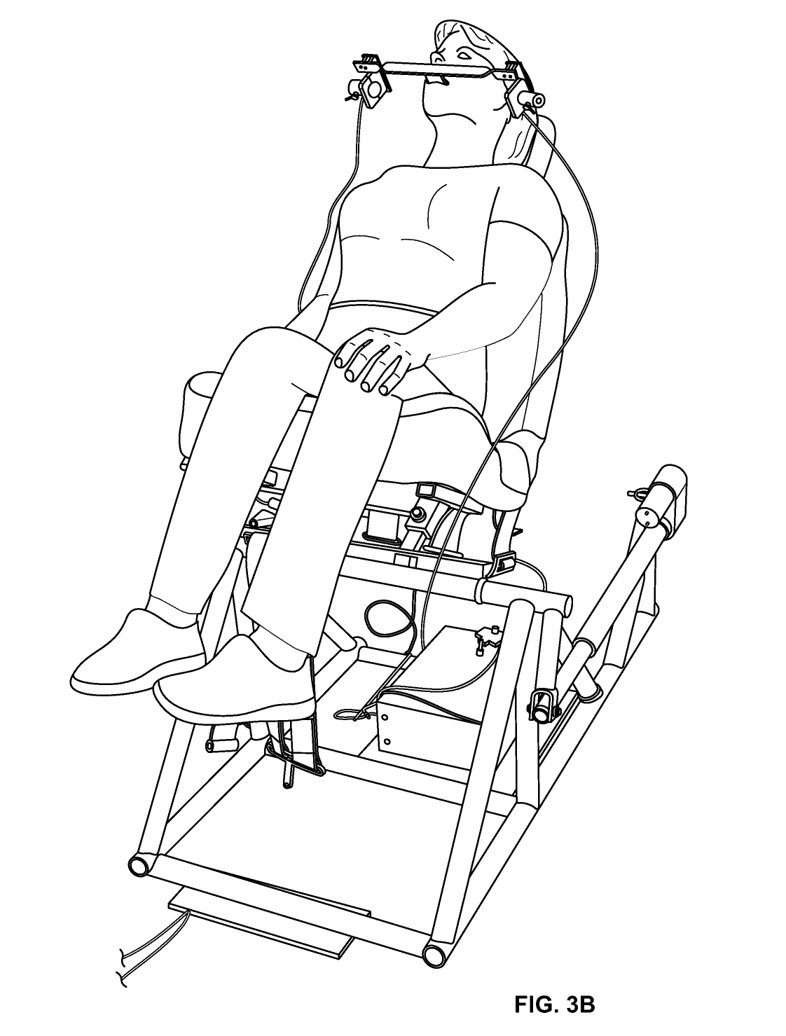

FIG. 3 shows an embodiment of a perspective view of a chair 300 that can move in a controlled range of motion that the patient sits on to measure the patient's skin firmness. After the adhesive dots 210 were placed on both sides of the face, the patient was secured in the motorized chair 300. To restrict body movement during the video process, the head was placed in one embodiment in a foam headrest 310 and the head was restricted from movement by securing a strap across the forehead. Straps 315 were also used to restrict torso movement.

In one embodiment, the chair 300 is a modified car seat attached to a custom design frame that includes a bell crank linkage to which a linear servo actuator is attached. The speed and direction of the to and fro motion of the linear servo actuator is controlled electronically (e.g., by a processor or computing device in communication with the chair 300). The chair 300 also contains an accelerometer that is placed in such a way that the tilt of the chair can be monitored in real time and displayed on a computer monitor or display screen (e.g., smartphone).

In addition, there are a plurality (e.g., two) digital cameras (e.g., camera 320) positioned so that they can simultaneously capture the movement of the patient's face. The digital cameras 320 may be stand-alone digital cameras or may be part of another device (e.g., part of a smartphone or tablet). In another embodiment, a helmet-CAM is used.

The chair is, for example, swept through a 60 degree arc from an upright position to fully laying back, and then returning to the initial seated position. During this time, both the video camera outputs and accelerometer display are captured as a single video clip. Post processing may be accomplished using specially designed software which allows one to visually display the trajectory of fiducial dots that have been placed in different spots on the cheek. In one embodiment, the software also provides various metrics on how these dots moved during the 60 degree sweep. Although a 60 degree sweep is described, any amount of sweep or arc may be utilized.

The videos can then be analyzed to measure displacement of the dots from their respective points of origin through each position for each age group. Differences in facial sagging and treatment differences are then captured between age groups. As an example, analysis was performed via object tracking using Image Pro Plus software (e.g., version 7) manufactured by Media Cybernetics, Inc. of Rockville, Md.

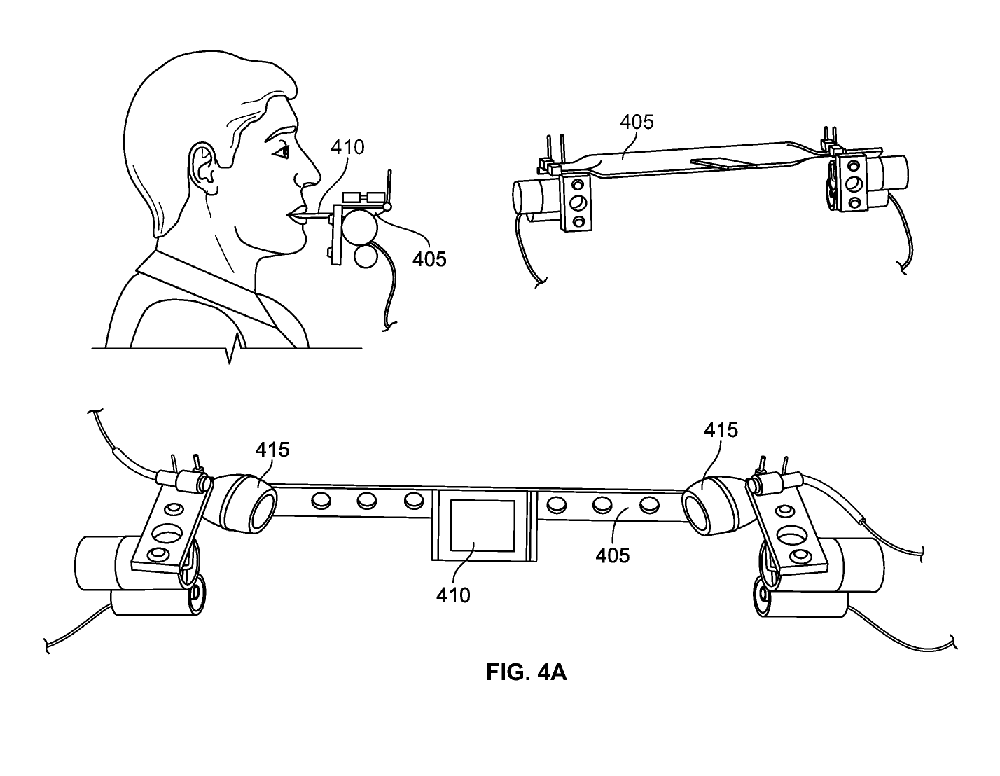

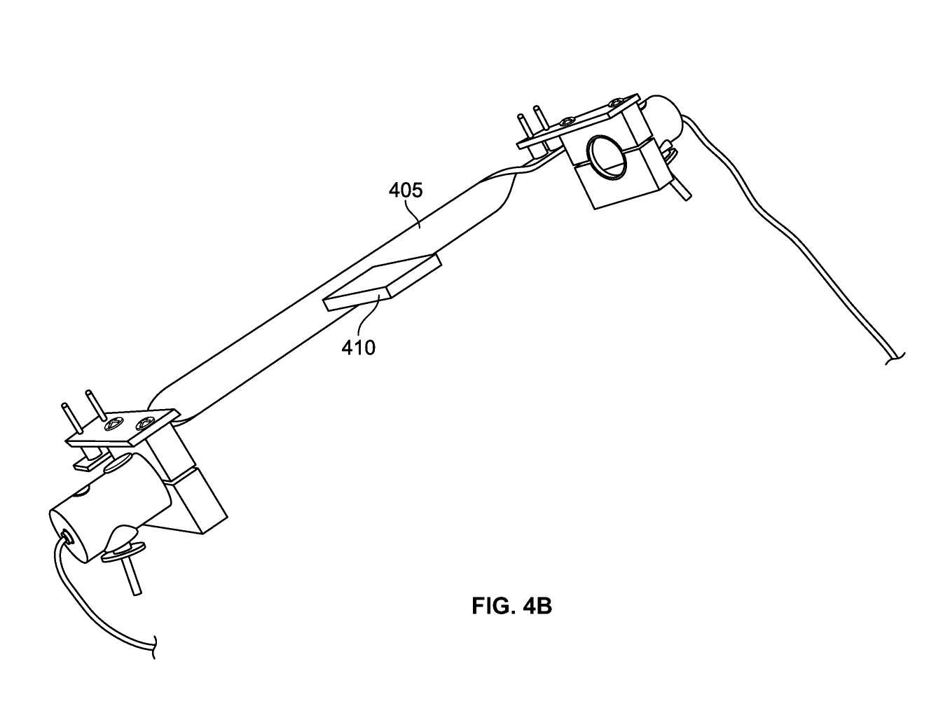

Referring to FIG. 4, in one embodiment, to reduce or eliminate the changes in the field of view as the patient's head was tilted either forward or backwards, the video cameras are mounted to a bar 405. By having the patient firmly engage (e.g., bite onto) a contact point (e.g., a plate) 410 in the center of this bar 405, the proper relationship between the cameras and the face can be maintained regardless of how the patient is being tilted. In one embodiment, the plate 410 is a bite plate customized for the patient. In another embodiment, the plate 410 is a general bite plate that can be used for any patient. In one embodiment, lights 415 are placed on the bar to reduce the apparent motion of the fiducial dots on the nose due to changes in illumination. The lights can be any type of bulbs, such as incandescent bulbs or halogen bulbs. The cameras may be placed on the bar additionally or alternatively to monitor the patient's face.

By simultaneously filming the left and right sides of the face during the same tilt excursion, differences in these two sides can be directly compared. Some older individuals can have pronounced differences between the left and right sides of their faces. This may be due to differences in chronic solar exposure.

As described above, the videos can be analyzed using a software routine for object tracking written in Image Pro Plus. This can enable one to determine the path that each of the individual dots moved as the patient was titled to different degrees.

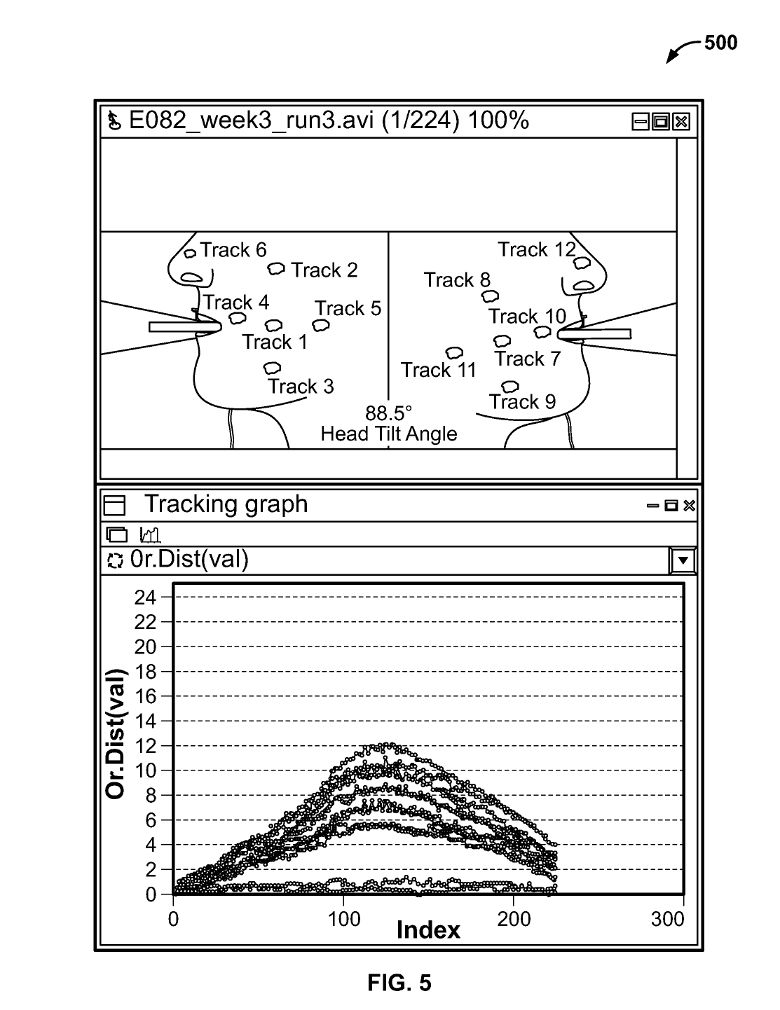

FIG. 5 shows a graphical representation 500 of the trajectory of each dot during the tilt excursion. The fiducial dots on the nose shows very little movement. This is expected since the skin is very firm in this region. This plot 500 also shows that there are differences in the degree and pattern of movement among the five target dots. The path is not purely linear but tends to be somewhat curved. The degree to which this occurs depends upon the position of the dot and more importantly the severity of sagging. Thus, previous methods based on simply measuring linear displacement in paired pre and post photos are likely underestimating the degree of facial sagging.

In one embodiment, since the tilt angle is continuously being read throughout the chair's excursion, a more detailed analysis can be performed of how changes in the direction of the gravitational pull can distort the face differently that might give clues to the underlying skin that is unique to that individual. This may provide some guidance to a plastic surgeon or a dermatologist using laser resurfacing to tone up the skin to enhance the outcome for that particular patient.

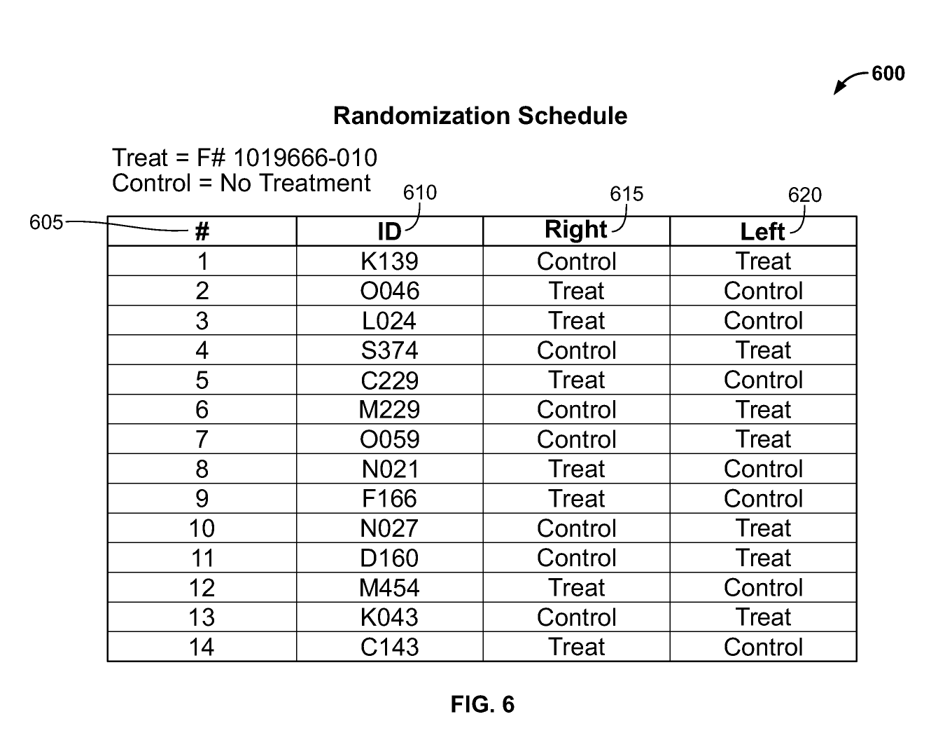

FIG. 6 is an embodiment of a tabular representation 600 of a randomization schedule. A count 605 is associated with a patient's ID number 610. The patient data is represented in table 600 with a right cheek column 615 being either Control or Treat and a left cheek column 620 being either Control or Treat. A Control entry means no treatment, and a Treat entry means application of a test product (e.g., Test Product F#1019666-010).

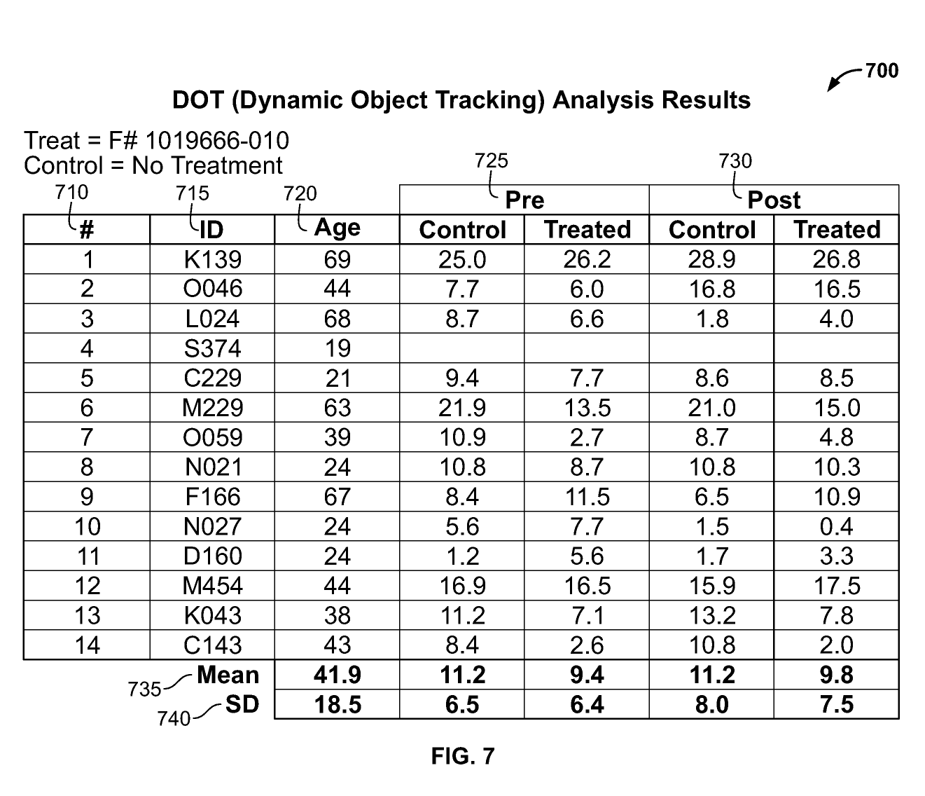

FIG. 7 is an embodiment of a tabular representation 700 of DOT analysis results of a demonstrative experiment for validating the effectiveness of the quantifying of skin firmness. This tabular representation 700 records the data obtained at baseline (prior to treatment) and approximately 10 minutes after treatment. The tabular representation 700 includes a count 710, a patient's ID number 715, and an age 720 of the patient. Each patient has a Pre column 725 representing the DOT analysis results for the patient before being moved in the chair 300 and a Post column 730 represent the DOT analysis results for the patient after being moved in the chair 300. The Pre column 725 and Post column 730 are divided into Control and Treated columns. A Control number represents the DOT analysis results without treatment, and a Treated number represents the DOT analysis results after application of a test product (e.g., Test Product F#1019666-010). The mean and standard deviation is also shown in a Mean row 735 and a SD row 740.

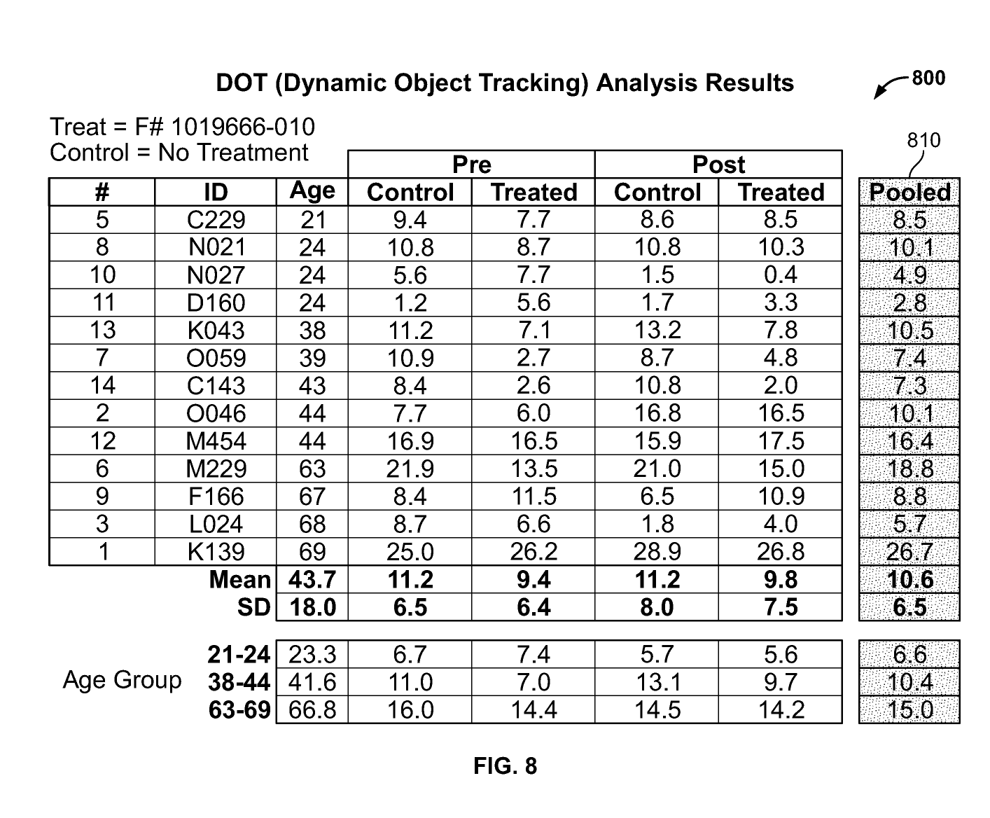

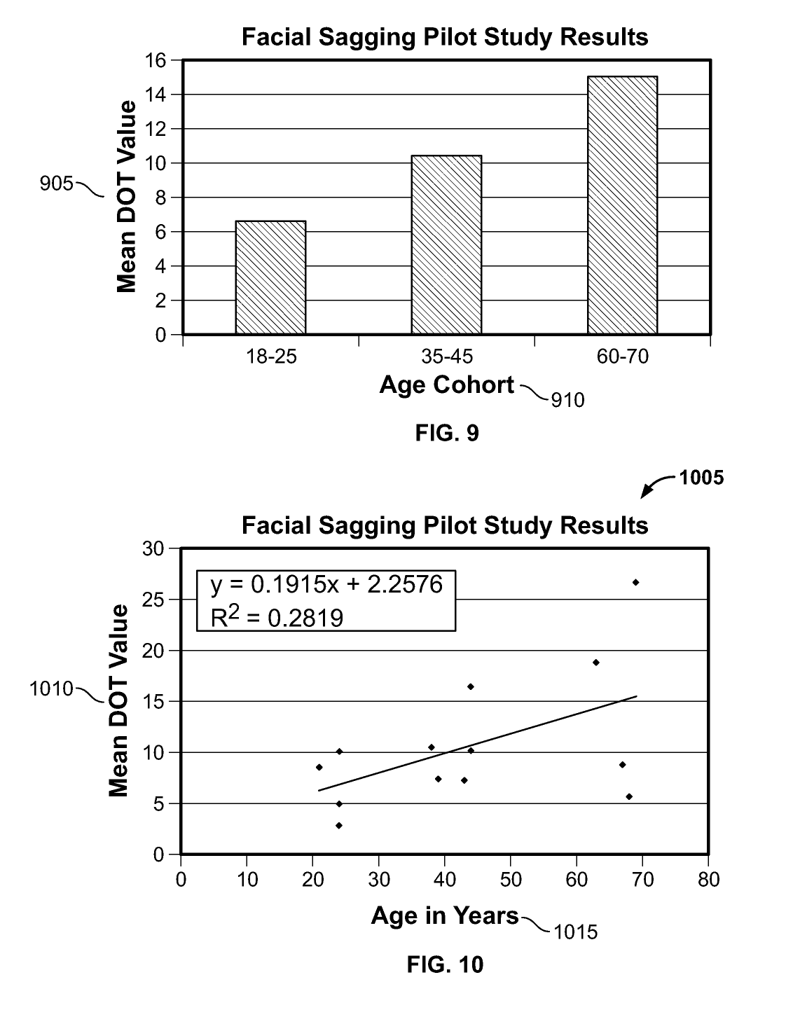

FIG. 8 shows an embodiment of the tabular representation of FIG. 7 ordered by age from youngest to oldest. In this table 800, a representative value for each individual was calculated by pooling their two baseline values prior to treatment and the post treatment value for the non-treated control (as shown in Pooled column 810). FIG. 9 is a graphical representation 900 of the mean DOT value 905 plotted against age cohort 910. FIG. 10 is a graphical representation 1005 of the mean DOT value 1010 plotted against age in years 1015. As shown in FIG. 9, the average values for each of the three age cohorts that participated increased with age. This was expected because the patients between ages of 18-25 were to have no apparent facial sagging, patients between the ages of 35-45 were to have mild-moderate facial sagging, and patients between the ages of 60-70 were to have moderate-severe facial sagging. As shown in FIG. 10, the correlation coefficient did show a positive correlation between age and facial sagging.

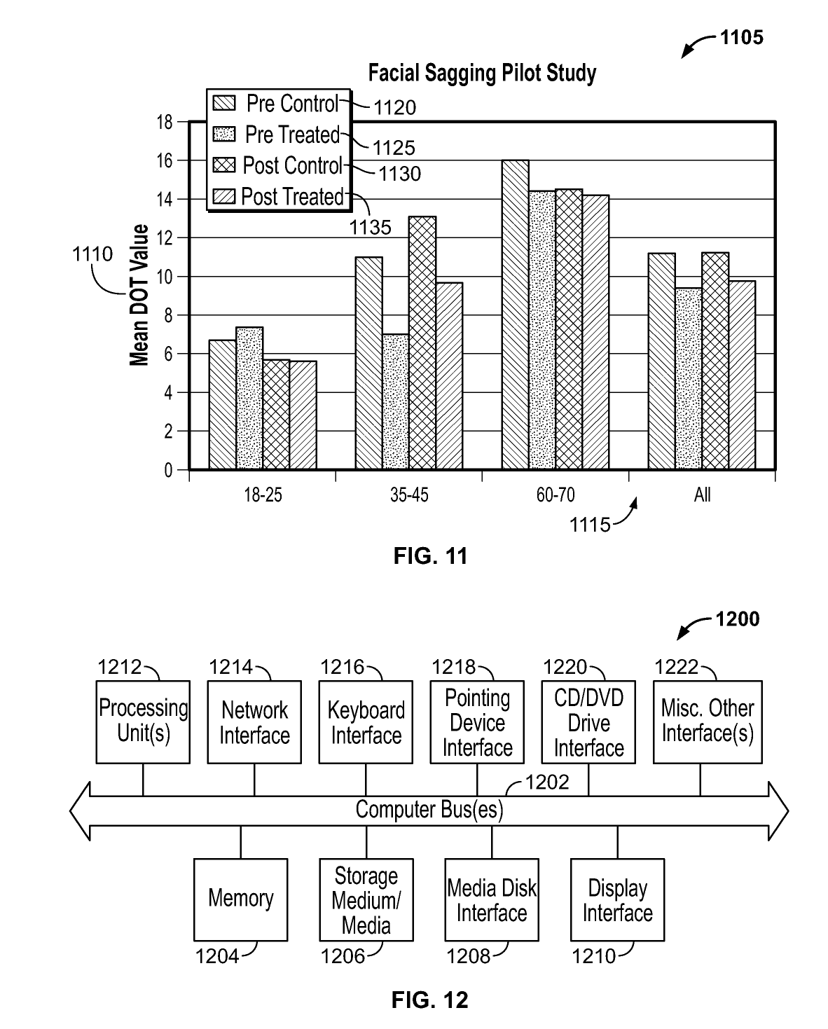

FIG. 11 shows an embodiment of a graphical representation 1105 of a demonstrative experiment for validating the effectiveness of the quantifying of skin firmness. Mean DOT value 1110 plotted against age cohort 1115 for pre control 1120, pre treated 1125, post control 1130, and post treated 1135 for Test Product F#1019666-010. The graphical representation 1105 is to determine whether treating with the test product would cause an immediate increase in facial firmness that could be detected. Here the expected result would be a decrease in the mean DOT value in the treated site relative to the non-treated control. Although plot 1105 does not show this, other test products and/or sample sizes may produce different results.

In another embodiment, to measure skin firmness, no dots are used. Instead, the amount of tightening of the skin of a patient due to a firming agent is monitored. This is monitored by reviewing and analyzing distortion of a reference grid over time. For example, instead of placing reference dots at fixed positions on the skin, a microprojector is used to place grid overlays onto the skin surface and then film how this pattern distorts as the firming agent acts to shrink the skin.

In one embodiment, strings can be glued to the skin surface. Strings can be pulled to distort the skin in a standardized fashion by applying a calculated degree of force. The movement of dots can be filmed during excursion.

FIG. 12 is a block diagram illustrating an internal architecture of an example of a computer or computing device, such as server computer and/or client device, in accordance with one or more embodiments of the present disclosure. A computer as referred to herein refers to any device with a processor capable of executing logic or coded instructions, and could be a server, personal computer, set top box, tablet, smart phone, pad computer or media device, to name a few such devices. As shown in the example of FIG. 12, internal architecture 1200 includes one or more processing units (also referred to herein as CPUs) 1212, which interface with at least one computer bus 1202. Also interfacing with computer bus 1202 are persistent storage medium/media 1206, network interface 1214, memory 1204, e.g., random access memory (RAM), run-time transient memory, read only memory (ROM), etc., media disk drive interface 1208 as an interface for a drive that can read and/or write to media including removable media such as floppy, CD-ROM, DVD, etc. media, display interface 1210 as interface for a monitor or other display device, keyboard interface 1216 as interface for a keyboard, pointing device interface 1218 as an interface for a mouse or other pointing device, CD/DVD drive interface 1220, and miscellaneous other interfaces 1222 not shown individually, such as parallel and serial port interfaces, a universal serial bus (USB) interface, and the like.

Memory 1204 interfaces with computer bus 1202 so as to provide information stored in memory 1204 to CPU 1212 during execution of software programs such as an operating system, application programs, device drivers, and software modules that comprise program code, and/or computer-executable process steps, incorporating functionality described herein, e.g., one or more of process flows described herein. CPU 1212 first loads computer-executable process steps from storage, e.g., memory 1204, storage medium/media 1206, removable media drive, and/or other storage device. CPU 1212 can then execute the stored process steps in order to execute the loaded computer-executable process steps. Stored data, e.g., data stored by a storage device, can be accessed by CPU 1212 during the execution of computer-executable process steps.

Persistent storage medium/media 1206 is a computer readable storage medium(s) that can be used to store software and data, e.g., an operating system and one or more application programs. Persistent storage medium/media 1206 can also be used to store device drivers, such as one or more of a digital camera driver, monitor driver, printer driver, scanner driver, or other device drivers, web pages, content files, playlists and other files. Persistent storage medium/media 1206 can further include program modules and data files used to implement one or more embodiments of the present disclosure.

For the purposes of this disclosure a computer readable medium stores computer data, which data can include computer program code that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other physical or material medium which can be used to tangibly store the desired information or data or instructions and which can be accessed by a computer or processor.

Internal architecture 1200 of the computer can include a microphone, video camera, TV/radio tuner, audio/video capture card, sound card, analog audio input with A/D converter, modem, digital media input (HDMI, optical link), digital I/O ports (RS232, USB, FireWire, Thunderbolt), and/or expansion slots (PCMCIA, ExpressCard, PCI, PCIe).

Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the user computing device or server or both. In this regard, any number of the features of the different embodiments described herein may be combined into single or multiple embodiments, and alternate embodiments having fewer than, or more than, all of the features described herein are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

While the system and method have been described in terms of one or more embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method comprising:
positioning a patient in a restraining device that can move in a controlled range of motion and that restricts movement of a patient, the patient having adhesive dots positioned at predetermined locations on the patient's body;
instructing, by a processor, said restricting device to move the patient at a predetermined sweep rate in a predetermined motion, wherein the predetermined motion comprises moving said restraining device along a predetermined plane;
obtaining, by a processor, a plurality of images during the predetermined motion;
analyzing, by a processor, the plurality of images to determine an amount of movement of the adhesive dots on the patient's body.

2. The method of claim 1, further comprising positioning the adhesive dots at the predetermined locations on the patient's body.

3. The method of claim 2, wherein the positioning of the adhesive dots at the predetermined locations on the patient's body further comprises positioning the adhesive dots on both left side and right side of the patient's face.

4. The method of claim 3, further comprising positioning the adhesive dots on the nose of the patient.

5. The method of claim 3, wherein the restraining device comprises a bar mounted on the restraining device, the bar comprising a contact point for the patient to engage to maintain a proper relationship between cameras and the patient's face regardless of how the restraining device is moved.

6. The method of claim 5, wherein the contact point is a bite plate.

7. The method of claim 1, further comprising projecting a grid on the patient's body.

8. A restraining device that can move in a controlled range of motion and that restricts movement of a patient, the restraining device comprising:
a processor;
a storage medium for tangibly storing thereon program logic for execution by the processor, the program logic comprising:
instructing logic executed by the processor for instructing the restraining device to move the patient at a predetermined sweep rate in a predetermined motion, the patient having adhesive dots positioned at predetermined locations on the patient's body;
obtaining logic executed by the processor to obtain a plurality of images during the predetermined motion; and
analyzing logic executed by the processor to analyze the plurality of images to determine an amount of movement of the adhesive dots on the patient's body.

9. The restraining device of claim 8, wherein the adhesive dots are positioned on both left side and right side of the patient's face.

10. The restraining device of claim 9, further comprising a plurality of cameras positioned to obtain the plurality of images.

11. The restraining device of claim 10, wherein the restraining device comprises a bar mounted on the restraining device, the bar comprising a contact point for the patient to engage to maintain a proper relationship between the plurality of cameras and the patient's face regardless of how the restraining device is moved.

12. The restraining device of claim 8, further comprising a modified car seat attached to a design frame that includes a bell crank linkage.

13. The restraining device of claim 12, further comprising a linear servo actuator attached to the bell crank linkage.

14. The restraining device of claim 13, wherein speed and direction of motion of the linear servo actuator is controlled electronically via the processor.

15. The restraining device of claim 8, further comprising an accelerometer enabling the predetermined motion of the chair to be monitored and displayed on a display screen.

16. The restraining device of claim 8, further comprising a projector in communication with the processor for projecting a grid on the patient's body before applying the adhesive dots.

17. A method comprising:
positioning adhesive dots at predetermined locations on a body of a patient;
obtaining, by cameras, a plurality of images of the adhesive dots; and
analyzing, by the computing device, the plurality of images to determine an amount of
movement of the adhesive dots on the patient's body.

18. A method comprising:
positioning a patient in a restraining means that can move in a controlled range of motion and that restricts movement of a patient, the patient having adhesive dots positioned at predetermined locations on the patient's body;
instructing, by a processor, said restricting means to move the patient at a predetermined sweep rate in a predetermined motion, wherein the predetermined motion comprises moving said restraining means along a predetermined plane;
obtaining, by the processor, a plurality of images during the predetermined motion;
analyzing, by the processor, the plurality of images to determine an amount of movement of the adhesive dots on the patient's body.

19. The method of claim 17, wherein the patient is instructed to move or attempt to move in a predetermined manner after the adhesive dots are positioned.

20. The method of claim 19, wherein the cameras obtain a plurality of images during the movement or attempted movement.
